(19) United States
(12) Patent Application Publication
(10) Pub. No.: US 2024/0122542 A1
(43) Pub. Date: Apr. 18, 2024

(54) WEARABLE DEVICE

(71) Applicant: Guardian Watch Inc. LLC, Bowling Green, OH (US)

(72) Inventors: Katie E. Wieber, Bowling Green, OH (US); Joseph C. Higgins, Bowling Green, OH (US)

(21) Appl. No.: 18/486,392

(22) Filed: Oct. 13, 2023

Related U.S. Application Data
(60) Provisional application No. 63/379,380, filed on Oct. 13, 2022.

(51) Int. Cl. A61B 5/00

(52) U.S. Cl. CPC A61B 5/681; A61B 5/747

(57) ABSTRACT

A wearable device may include a user interface, a processor in communication with a memory having executable instructions, an application, and at least sensor configured to monitor and/or track health data of the user. The processor executes the instructions to identify and transmit abnormal health events to initiate a response from one or more predetermined contacts and/or services.

### CROSS-REFERENCE TO RELATED APPLICATION

This application claims the benefit of U.S. Provisional Patent Application Ser. No. 63/379,380, filed Oct. 13, 2022, the entirety of which is herein incorporated by reference.

### FIELD

The disclosure relates to a wearable device, and more particularly to a wearable device configured to initiate a response to an abnormal health event.

### BACKGROUND

Currently technology integrated with various health tools is becoming a very popular trend within the healthcare industry and is increasingly being used on a more regular basis. Wearable devices are one such category of technology. Many of the wearable devices that are providing a plethora of health data that may be used to inform both personal and clinical decisions for consumers utilize the growing roster of available tools. Generally, wearable devices with health tools may measure the heart rate (HR), heart rate variability (HRV), blood oxygen saturation, temperature, motion, and/or other biological information of the user via noninvasive methods.

In one main application field, health tools are integrated into a smart watch or bracelet. However, the smart watch or bracelet is designed to only transmit communications to the user. In another application field, a pulse oximeter may be used to measure health data of the user at the fingertip. However, it is inconvenient and not stable for long-term wear on the fingertip, especially during daily activities.

Accordingly, there is a need for a wearable device to be used for an extended period of time for health data measurements and monitoring status of the user without causing any inconvenience or discomfort to the user, which is configured to transmit communications to at least one predetermined contact to initiate a response to an abnormal health event.

### SUMMARY

In concordance and agreement with the presently described subject matter, a wearable device for health data measurement and monitoring status of a user, which is configured to transmit communications to at least one predetermined contact to initiate a response to an abnormal health event, has surprisingly been discovered.

In one embodiment, a wearable device, comprises: a case; at least one sensor at least partially disposed in the case and configured to detect health data of a user; and at least one processor in communication with the at least one sensor, the at least one processor configured to receive the health data and transmit a communication to initiate a response from at least one predetermined contact based upon the health data.

As aspects of some embodiments, the at least one processor is configured to utilize at least one algorithm parameter to determine an occurrence of an abnormal health event.

As aspects of some embodiments, the at least one algorithm parameter is based upon a predisposition of a user.

As aspects of some embodiments, the at least one processor is in communication with at least one storage device configured to store at least one of the health data, the at least one algorithm parameter, and personal information of the at least one predetermined contact.

As aspects of some embodiments, the wearable device is configured to use machine learning to at least one of generate and adjust the at least one algorithm parameter.

As aspects of some embodiments, the at least one sensor is configured to detect the health data at one or more of a predetermined frequency, a customizable frequency, and on demand.

As aspects of some embodiments, the at least one processor is configured to generate at least one alert communication via an input and output system of the wearable device during an occurrence of an abnormal health event.

As aspects of some embodiments, the wearable device further comprises a graphical user interface in communication with the at least one processor and an input and output system of the wearable device.

As aspects of some embodiments, the communication transmitted by the at least one processor is configured to bypass at least one setting of an electronic device of the at least one predetermined contact.

As aspects of some embodiments, the at least one sensor is configured to detect a human connection.

In another embodiment, a method of initiating a response, comprises steps of: providing a wearable device including at least one sensor and at least one processor; detecting health data of a user via the at least one sensor; and transmitting a communication to initiate a response from at least one predetermined contact based upon the health data.

As aspects of some embodiments, the method further comprises the step of determining, via the at least one processor, an occurrence of an abnormal health event using at least one algorithm parameter.

As aspects of some embodiments, the method further comprises the step of storing at least one of the health data, at least one algorithm parameter, and personal information of the at least one predetermined contact.

As aspects of some embodiments, the method further comprises the step of using machine learning to at least one of generate and adjust at least one algorithm parameter.

As aspects of some embodiments, the method further comprises the step of outputting at least one alert communication via an input and output system of the wearable device during an occurrence of an abnormal health event.

As aspects of some embodiments, the method further comprises the step of detecting detect a human connection using the at least one sensor.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1C are schematic representations of various wearable devices according to an embodiment of the present disclosure;

FIG. 2 is a schematic representation of an exemplary embodiment of the wearable device illustrating components thereof;

FIG. 3 is a flow diagram illustrating an initialization process of the wearable devices of FIGS. 1A-2;

FIG. 4 is a flow diagram illustrating a notification process of the wearable devices of FIGS. 1A-2; and

FIG. 5 is a flow diagram illustrating an operation process of the wearable devices of FIGS. 1A-2.

### DETAILED DESCRIPTION

The following description of technology is merely exemplary in nature of the subject matter, manufacture and use of one or more present disclosures, and is not intended to limit the scope, application, or uses of any specific present disclosure claimed in this application or in such other applications as may be filed claiming priority to this application, or patents issuing therefrom. Regarding methods disclosed, the order of the steps presented is exemplary in nature, and thus, the order of the steps may be different in various embodiments. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” may encompass both an orientation of above and below. The device may be otherwise oriented (rotated 90 degrees or at other orientations) and the spatially relative descriptors used herein interpreted accordingly.

Apparatuses, machine-readable media, and methods related to monitoring health data and associated emergency assistance responses are described. Health related events may occur at any time. For instance, falls, blood glucose level spikes, blood pressure issues, etc. may occur when someone is alone or unable to get to a telephone. Some alert systems allow for a user to push a button and reach emergency services if the user needs help. However, such systems may not allow for automatic alerts (e.g., with little or no user interaction), tracking of health events, or communication with other health monitoring sensors, such as blood glucose monitors, heartrate monitors, or blood pressure monitors, among others.

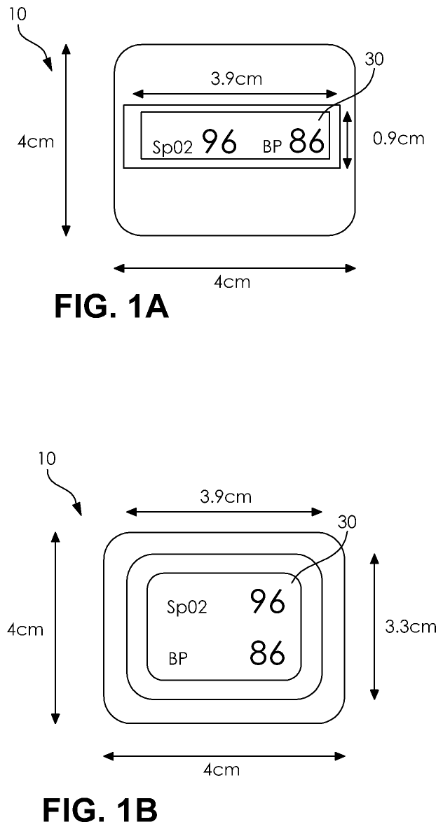

FIGS. 1A-1C show various wearable devices 10 in accordance with an embodiment of the present disclosure. The wearable device 10 may be, but is not limited to, a watch, a bracelet, a necklace, a ring, a wearable monitor, pendant, waterproof device, or other device that may be wearable. The wearable device 10 may include components such as, at least one of each of a processing device or processor 12, and a memory device or memory 14 for processing use, such as random access memory (RAM), and read-only memory (ROM). The wearable device 10 further includes a database 16 including at least one of a non-transitory storage medium, such as a microdrive, for long-term, intermediate-term, and short-term storage of computer-readable instructions 18 for execution by the processor 12. For example, the instructions 18 can include instructions for an operating system and various applications or programs 20, of which the application 20 is represented as a particular example. The database 16 can store various other data items 22, which can include, as non-limiting examples, cached data, user files such as those for pictures, audio and/or video recordings, files downloaded or received from other devices, and other data items preferred by the user or required or related to any or all of the applications or programs 20. The wearable device 10 further includes at least sensor 24 configured to monitor and/or track health data of the user. Although the following descriptions refer to a processor 12 and a memory 14, the descriptions hereinafter may also apply to multiple processors 12 and multiple memory devices 14. In such examples, the instructions 18 may be distributed (e.g., stored) across multiple memory devices and the instructions 18 may be distributed (e.g., executed by) across multiple processors 12.

The memory 14 is operatively coupled to the processor 12. As used herein, the memory 14 includes any computer readable medium to store data, code, or other information. The memory 14 may be an electronic, magnetic, optical, or other physical storage device that stores executable instructions 18. Thus, the memory 14 may be, for example, non-volatile or volatile memory. For example, non-volatile memory may provide persistent data by retaining written data when not powered, and non-volatile memory types may include NAND flash memory, NOR flash memory, read only memory (ROM), Electrically Erasable Programmable ROM (EEPROM), Erasable Programmable ROM (EPROM), and Storage Class Memory (SCM) that may include resistance variable memory, such as phase change random access memory (PCRAM), three-dimensional cross-point memory, resistive random access memory (RRAM), ferroelectric random access memory (FeRAM), magnetoresistive random access memory (MRAM), and programmable conductive memory, among other types of memory. Volatile memory may require power to maintain its data and may include random-access memory (RAM), dynamic random-access memory (DRAM), and static random-access memory (SRAM), among others.

In some examples, the memory 14 may be a non-transitory MRM comprising Random Access Memory (RAM), an Electrically-Erasable Programmable ROM (EEPROM), a storage drive, an optical disc, and the like. The memory 14 may be disposed within the wearable device 10 and/or other computing device. In this example, the executable instructions 18 may be “installed” on the wearable device 10. Additionally, and/or alternatively, the memory 14 may be a portable, external or remote storage medium, for example, that allows the system to download the instructions 18 from the portable/external/remote storage medium. In this situation, the executable instructions 18 may be part of an “installation package”. As described herein, the memory 14 may be encoded with executable instructions 18 for an emergency assistance response.

According to various embodiments, the memory 14 and database 16 may be combined into a single storage medium. The memory 14 and database 16 can store any of a number of applications 20 which comprise computer-executable instructions 18 and code executed by the processor 12 to implement the functions of the wearable device 10 described herein. For example, the memory 14 may include such applications 20 as a conventional web browser application. These applications 20 also typically provide a graphical user interface (GUI) on a display 30 that allows the user to communicate with the wearable device 10, and/or other devices or systems. In some embodiments, when the user decides to participate in an application 20, the user may download or otherwise obtain the application 20 from a distinct application server. In other embodiments, the user interacts with the application 20 via a web browser application.

The processor 12, and other processors described herein, generally include circuitry for implementing communication and/or logic functions of the wearable device 10. For example, the processor 12 may include a digital signal processor, a microprocessor, and various analog to digital converters, digital to analog converters, and/or other support circuits. Control and signal processing functions of the wearable device 10 are allocated between these devices according to their respective capabilities. The processor 12 thus may also include the functionality to encode and interleave messages and data prior to modulation and transmission. The processor 12 can additionally include an internal data modem. Further, the processor 12 may include functionality to operate one or more software programs, which may be stored in the memory 14, or in the database 16. For example, the processor 12 may be capable of operating a connectivity program, such as a web browser application. The web browser application may then allow the wearable device 10 to transmit and receive web content, such as, for example, location-based content and/or other web page content, according to a Wireless Application Protocol (WAP), Hypertext Transfer Protocol (HTTP), and/or the like.

The memory 14 and database 16 can each also store any of a number of pieces of information, and data, used by the wearable device 10 and the applications and devices that facilitate functions of the wearable device 10, or are in communication with the wearable device 10, to implement the functions described herein and others not expressly described. For example, the database 16 may include such data as user authentication information, etc.

The processor 12, in various examples, can operatively perform calculations, can process instructions 18 for execution, and can manipulate information. The processor 12 can execute machine-executable instructions 18 stored in the database 16 and/or memory 14 to thereby perform methods and functions as described or implied herein, for example by one or more corresponding flow charts expressly provided or implied as would be understood by one of ordinary skill in the art to which the subject matters of these descriptions pertain. The processor 12 can be or can include, as non-limiting examples, a central processing unit (CPU), a microprocessor, a graphics processing unit (GPU), a microcontroller, an application-specific integrated circuit (ASIC), a programmable logic device (PLD), a digital signal processor (DSP), a field programmable gate array (FPGA), a state machine, a controller, gated or transistor logic, discrete physical hardware components, and combinations thereof. In some embodiments, particular portions or steps of methods and functions described herein are performed in whole or in part by way of the processor 12, while in other embodiments methods and functions described herein include cloud-based computing in whole or in part such that the processor 12 facilitates local operations including, as non-limiting examples, communication, data transfer, and user inputs and outputs such as receiving commands from and providing displays to the user.

The wearable device 10, as illustrated, includes an input and output system 32, referring to, including, or operatively coupled with, one or more user input devices and/or one or more user output devices, which are operatively coupled to the processor 12. The input and output system 32 may include input/output circuitry that may operatively convert analog signals and other signals into digital data, or may convert digital data to another type of signal. For example, the input/output circuitry may receive and convert physical contact inputs, physical movements, or auditory signals (e.g., which may be used to authenticate a user) to digital data. Once converted, the digital data may be provided to the processor 12. The input and output system 32 may also include the display 30 (e.g., a liquid crystal display (LCD), light emitting diode (LED) display, or the like), which can be, as a non-limiting example, a presence-sensitive input screen (e.g., touch screen or the like) of the wearable device 10, which serves both as an output device, by providing graphical and text indicia and presentations for viewing by one or more user, and as an input device, by providing virtual buttons, selectable options, a virtual keyboard, and other indicia that, when touched, control the wearable device 10 by user action. The user output devices include a speaker or other audio device. The user input devices, which allow the wearable device 10 to receive data and actions such as button manipulations and touches from a user, may include any of a number of devices allowing the wearable device 10 to receive data from a user, such as a keypad, keyboard, touch-screen, touchpad, microphone, mouse, joystick, other pointer device, button, soft key, infrared sensor, and/or other input device(s). The input and output system 32 may also include a camera, such as a digital camera.

Further non-limiting examples of input devices and/or output devices include, one or more of each, any, and all of a wireless or wired keyboard, a mouse, a touchpad, a button, a switch, a light, an LED, a buzzer, a bell, a printer and/or other user input devices and output devices for use by or communication with the user in accessing, using, and controlling, in whole or in part, the wearable device 10. Inputs by one or more users can thus be made via voice, text or graphical indicia selections.

The input and output system 32 may also be configured to obtain and process various forms of authentication via an authentication system to obtain authentication information of a user. Various authentication systems may include, according to various embodiments, a recognition system that detects biometric features or attributes of a user such as, for example fingerprint recognition systems and the like (hand print recognition systems, palm print recognition systems, etc.), iris recognition and the like used to authenticate a user based on features of the user's eyes, facial recognition systems based on facial features of the user, DNA-based authentication, or any other suitable biometric attribute or information associated with a user. Additionally or alternatively, voice biometric systems may be used to authenticate a user using speech recognition associated with a word, phrase, tone, or other voice-related features of the user. Alternate authentication systems may include one or more systems to identify a user based on a visual or temporal pattern of inputs provided by the user. For instance, the user device may display, for example, selectable options, shapes, inputs, buttons, numeric representations, etc. that must be selected in a pre-determined specified order or according to a specific pattern. Other authentication processes are also contemplated herein including, for example, email authentication, password protected authentication, device verification of saved devices, code-generated authentication, text message authentication, phone call authentication, etc. The wearable device 10 may enable users to input any number or combination of authentication systems.

The wearable device 10 may also include a positioning device 34, which can be for example a global positioning system device (GPS) configured to be used by a positioning system to determine a location of the wearable device 10. For example, the positioning device 34 may include a GPS transceiver. In some embodiments, the positioning device 34 includes an antenna, transmitter, and receiver. For example, in one embodiment, triangulation of signals may be used to identify the approximate location of the wearable device 10. In other embodiments, the positioning device 34 includes a proximity sensor or transmitter, such as an RFID tag, that can sense or be sensed by devices known to be located proximate another location to determine that the wearable device 10 is located proximate these known devices.

In the illustrated example, a system intraconnect 36, connects, for example electrically, the various described, illustrated, and implied components of the wearable device 10. The intraconnect 36, in various non-limiting examples, can include or represent, a system bus, a high-speed interface connecting the processor 12 to the memory 14, individual electrical connections among the components, and electrical conductive traces on a motherboard common to some or all of the above-described components of the wearable device 10. As discussed herein, the system intraconnect 36 may operatively couple various components with one another, or in other words, electrically connects those components, either directly or indirectly—by way of intermediate component(s)—with one another.

The wearable device 10 includes a communication interface 38, by which the wearable device 10 communicates and conducts transactions with other devices and systems. The communication interface 38 may include digital signal processing circuitry and may provide two-way communications and data exchanges, for example wirelessly, and for an additional or alternative example, via wired or docked communication by mechanical electrically conductive connector. Communications may be conducted via various modes or protocols, of which GSM voice calls, SMS, EMS, MMS messaging, TDMA, CDMA, PDC, WCDMA, CDMA2000, and GPRS, are all non-limiting and non-exclusive examples. Thus, communications can be conducted, for example, via the wireless communication device, which can be or include a radio-frequency transceiver, a Bluetooth device, Wi-Fi device, a Near-field communication device, and other transceivers. In addition, GPS (Global Positioning System) may be included for navigation and location-related data exchanges, ingoing and/or outgoing. Communications may also or alternatively be conducted via the connector for wired connections such by USB, Ethernet, and other physically connected modes of data transfer.

The processor 12 is configured to use the communication interface 38 as, for example, a network interface to communicate with one or more other devices on a network. In this regard, the communication interface 38 utilizes the wireless communication device as an antenna operatively coupled to a transmitter and a receiver (together a “transceiver”) included with the communication interface 38. The processor 12 is configured to provide signals to and receive signals from the transmitter and receiver, respectively. The signals may include signaling information in accordance with the air interface standard of the applicable system of a wireless telephone network. In this regard, the wearable device 10 may be configured to operate with one or more air interface standards, communication protocols, modulation types, and access types. By way of illustration, the wearable device 10 may be configured to operate in accordance with any of a number of first, second, third, fourth, fifth-generation communication protocols and/or the like. For example, the wearable device 10 may be configured to operate in accordance with second-generation (2G) wireless communication protocols IS-136 (time division multiple access (TDMA)), GSM (global system for mobile communication), and/or IS-95 (code division multiple access (CDMA)), or with third-generation (3G) wireless communication protocols, such as Universal Mobile Telecommunications System (UMTS), CDMA2000, wideband CDMA (WCDMA) and/or time division-synchronous CDMA (TD-SCDMA), with fourth-generation (4G) wireless communication protocols such as Long-Term Evolution (LTE), fifth-generation (5G) wireless communication protocols, Bluetooth Low Energy (BLE) communication protocols such as Bluetooth 5.0, ultra-wideband (UWB) communication protocols, and/or the like. The wearable device 10 may also be configured to operate in accordance with non-cellular communication mechanisms, such as via a wireless local area network (WLAN) or other communication/data networks.

The communication interface 38 may also include a healthcare network interface. The healthcare network interface may include software, such as encryption software, and hardware, such as a modem, for communicating information to and/or from one or more devices on a network. For example, the wearable device 10 may be configured so that it can be used as a healthcare portal by, for example, wirelessly communicating healthcare information or other information to a terminal of the network. Such communication could be performed via transmission over a wireless communication protocol such as the Near-field communication protocol.

The wearable device 10 further includes a power source 40, such as a battery, for powering various circuits and other devices that are used to operate the wearable device 10. Embodiments of the wearable device 10 may also include a clock or other timer configured to determine and, in some cases, communicate actual or relative time to the processor 12 or one or more other devices. For further example, the clock may facilitate timestamping transmissions, receptions, and other data for security, authentication, logging, polling, data expiry, and forensic purposes.

The wearable device 10 may be in communication, for example via the communication interface 38, with associated devices and/or systems (e.g., mobile device of at least one predetermined contact (e.g., an emergency contact), an automated, manual, and/or a third-party communications center, etc.), a database (e.g., a cloud storage service), or any combination thereof to work together as an emergency assistance response system (EARS) to appropriately respond to an emergency or potential emergency. For instance, the user of the wearable device 10 may experience an abnormal health event, and the wearable device 10 including the EARS may determine and/or initiate an appropriate response. The instructions 18 may be executable to identify and transmit, via the processor 12, output data representative of abnormal health events based on the health data and determine a response to the abnormal health event based on a reply or non-reply from the user via the input and output system 32 and/or display 30, and transmit a signal via the communication interface 38 to the EARS to initiate a response. The wearable device 10 may transmit across the system intraconnect 36 the health data to the database 16 for storage and/or later retrieval. In some embodiments, the wearable device 10 may also transmit the health data to other storage devices and systems via the communication interface 38.

In certain circumstances, the wearable device 10 may further initiate the positioning device 34 so that the wearable device 10 may be located by the EARS (e.g., for deployment of at least one predetermined contact and/or services).

In some embodiments, the wearable device 10 and/or the application 20 may be accessed via the GUI on the display 30 or other computing device, and data may be manually input, for example using the input and out system 32, therein. For instance, the user may input personal health data such as a desired ambulance service, insurance information, health information (e.g., weight, height, allergies, normal oxygen saturation levels/ranges, normal heart rates/ranges, normal blood pressure levels/ranges, disorders, diagnosis, etc.), and the like, for example, into the wearable device 10 such that the personal health data may be used to generate/define algorithm parameters available to the EARS and/or a monitoring center to determine whether an abnormal health event (e.g. a fall, an overdose, a heart attack, a seizure, and the like) has occurred and aid in decision making for an emergency assistance response decision for or with the user.

In certain embodiments, the personal health data and/or the algorithm parameters may be stored in the database 16 and adjusted by the user and/or the wearable device 10 based upon a predisposition of the user. It is understood that the wearable device 10 and/or application 20 may include and/or utilize a trained machine learning model (e.g., artificial intelligence (AI)), algorithms, lookup tables, or a combination thereof, required for automatic adjustment of certain personal health data and/or algorithm parameters of the application 20. For example, normal oxygen saturation range of about 97% to about 100% may be manually adjusted by the user, using the input and output system 32, and/or automatically adjusted to a range of about 88% to about 92% by the wearable device 10 based upon personal health data received that indicates the user has chronic obstructive pulmonary disease (COPD).

The user, via the input and output system 32, may also input contact information of at least one predetermined contact, such as name and telephone number, for example, into the wearable device 10 and/or the application 20. The wearable device 10 and/or the application 20 may be configured to store the contact information in the database 16 and may accessed/used by the EARS when an abnormal health event occurs. In some embodiments, each of the predetermined contacts may receive a communication from the wearable device 10 and/or the application 20, via the communication interface 38, requesting acceptance as being one of the predetermined contacts of the user and authorization that their contact information may be stored in the database 16 and used by the EARS. In some embodiments, each of the predetermined contacts may receive a communication from the wearable device 10 and/or the application 20, via the communication interface 38, requesting approval as being removed as one of the predetermined contacts of the user and authorization that their contact information may be deleted from the database 16.

In some embodiments, a user who experiences an abnormal health event, while wearing the wearable device 10, may activate the wearable device 10 using the input and output system 32 (e.g., press a button), resulting in the EARS contacting the predetermined contacts and/or the monitoring center. The monitoring center may be a staffed monitoring center (e.g., staffed 24 hours per day, 7 days per week) to receive communications from the wearable device 10 and/or the application 20, via the communication interface 38. Decisions at the monitoring center may be made by trained and/or certified responders. In other embodiments, the EARS, without any input from the user, may directly notify the predetermined contacts and/or the monitoring center.

In certain embodiments, the at least one sensor 24 of the wearable device 10 may be configured to monitor present health data. The wearable device 10 and/or the application 20 may receive/collect the present health data from the at least one sensor 24. Examples may include, but are not limited to, oxygen saturation level sensors, heartrate sensors, blood pressure sensors, glucose sensors, kidney function sensors, respiratory sensors, insulin pump sensors, and/or temperature sensors, among others. The at least one sensor 24 may be integrated into the wearable device 10 (e.g., an integrated oxygen saturation level sensor, a heartrate sensor, a temperature sensor, etc.). The at least one sensor 24 may transmit the present health data to the application 20 or the wearable device 10, or both. The wearable device 10 may be configured to monitor and/or collect the present health data at a predetermined frequency (e.g., every 15 seconds), a customizable frequency (e.g., every 5 minutes between 8 a.m. and 5 p.m), and/or on demand by the user. It is understood that the collected present health data may be transmitted from the wearable device 10 and/or the application 20 to the database 16 for further storage. The wearable device 10 and/or the application 20 may be further configured to provide at least one alert communication (e.g., audible, haptic, and/or visual alert), for example via the input and output system 32, to the user and/or those persons within earshot of the wearable device 10 when the wearable device 10 and/or application 20, using the stored health data and/or the algorithm parameters and the collected health data from the at least one sensor 24 (e.g., a comparison of the collected health data to the stored health data and/or the algorithm parameters), detects an abnormal health event (e.g., an oxygen saturation level lower than the normal oxygen saturation level or outside the range stored in the database). The abnormal health event may be tracked, recorded, and saved in the application 20 and/or the database 16 for future retrieval and reference.

In some embodiments, when the at least one alert communication occurs, the user may be permitted to dismiss/deactivate the at least one alert communication within a threshold time period prior to the EARS initiating the emergency response. This may include initiating the input and output system 32 (e.g., pushing a button, tapping an icon on the user interface of the wearable device 10, or some other triggering of a particular input) on the wearable device 10. The dismissal by the user may act as confirmation that no help may be requested via the communication interface 38, or a separate confirmation may be received at the wearable device 10 that the user does not need emergency assistance.

In other embodiments, the user may dismiss/deactivate the at least one alert communication within the threshold time, but the wearable device 10 may be configured to also permit the user to indicate that emergency assistance may be needed, the predetermined contacts and/or services may then be notified by the EARS via the communication interface 38. In certain embodiments, if the wearable device 10 does not receive input from the user to dismiss/deactivate the at least one alert communication after the threshold time period, the EARS may then contact the predetermined contacts and/or the monitoring center. For instance, a telephone number of one or more of the predetermined contacts previously stored in the database 16 may be called or a text message may be sent. In some embodiments, the EARS simultaneously contacts each and every one of the predetermined contacts stored in the database 16. In other embodiments, a priority of the predetermined contacts may be assigned by the user and stored in the database 16, and the EARS consecutively contacts the predetermined contacts based upon such priority.

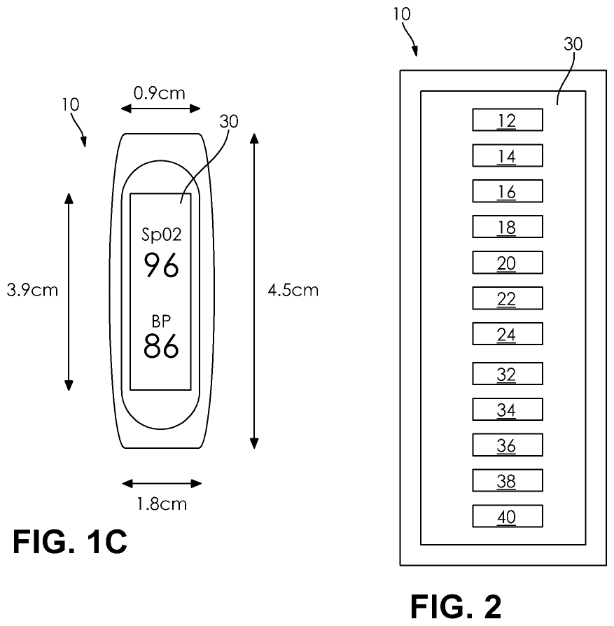

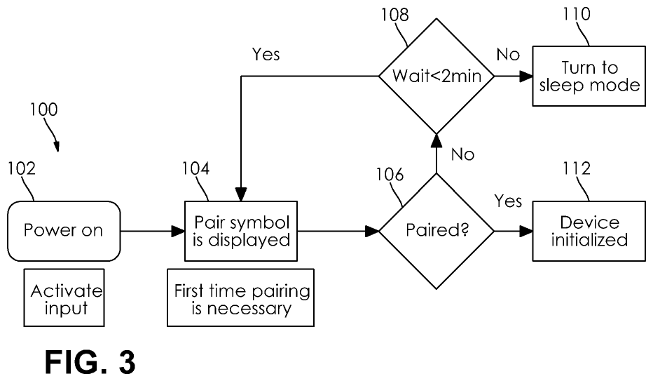

FIG. 3 is a flow diagram representing an exemplary method 100 of an initialization process in accordance with various embodiments of the present disclosure. The method 100 may be performed by the wearable device 10 described with respect to FIGS. 1A-2.

At step 102, the wearable device 10 is powered on/activated. In some embodiments, the wearable device 10 is activated by initiating the input and output system 32 (e.g., pushing a button, tapping an icon on the GUI of the wearable device 10, or some other triggering of a particular input on the wearable device 10). At step 104, a pair symbol is shown on the display 30 on the wearable device 10. When the wearable device 10 has not been previously paired, a first time pairing of the wearable device 10 is necessary. If the wearable device 10 at step 106 is not paired, a duration period runs at step 108 and compared to a duration period threshold. In some embodiments, the duration period threshold is less than 2 minutes. It is understood that the duration period threshold may be any duration of time as desired. In the event that the duration period is less than the duration period threshold, then step 104 is repeated. In the event that the duration period is at least the duration period threshold, then the wearable device 10 is turned to sleep mode at step 110. If the wearable device 10 at step 106 is paired, the wearable device 10 is initialized at step 112.

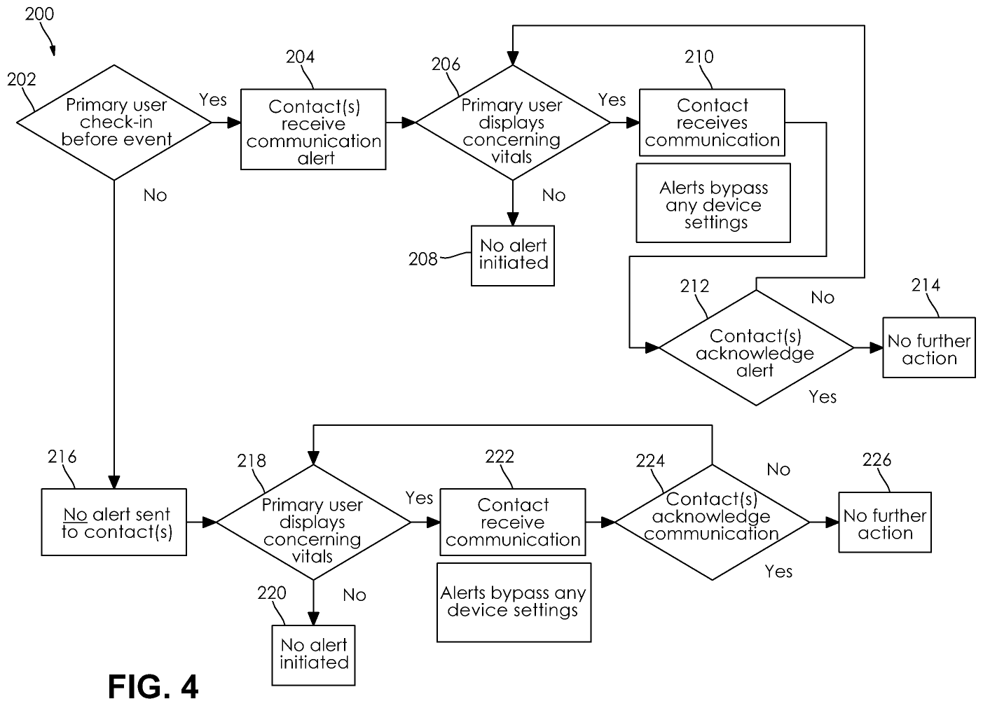

FIG. 4 is a flow diagram representing an exemplary method 200 of a notification process in accordance with various embodiments of the present disclosure. The method 200 may be performed by the wearable device 10 described with respect to FIGS. 1A-2.

At step 202, the method 200 may include a check-in of the user of the wearable device 10. When the check-in occurs at step 202, the at least one predetermined contact receives at least one alert communication (e.g., a text, email, telephone call, etc.), via the communication device interface 38, from the wearable device 10 and/or the application 20 at step 204. As step 206, the at least one sensor 24 monitors and/or senses the present health data of the user. If there is no abnormal health event, at step 208, the wearable device 10 and/or the application 20 does not generate and/or transmit the at least one alert communication. Contrarily, at step 210, when an abnormal health event occurs, the wearable device 10 and/or the application 20 generates and/or transmits the at least one alert communication to the at least one predetermined contact, via the communication interface 38. In certain embodiments, the at least one alert communication bypasses any device settings (e.g., silence and/or low volume) of the electronic device or other computing device of the at least one predetermined contact. At step 212, the at least one predetermined contact may be requested to acknowledge receipt of the at least one alert communication. If the at least one predetermined contact does not acknowledge receipt, step 206 is repeated. At step 214, when the at least one predetermined contact acknowledges receipt, no further action is taken.

When the check-in at step 202 does not occur, at least one predetermined contact does not receive at least one alert communication (e.g., a text, email, telephone call, etc.) from the wearable device 10 and/or the application 20 at step 216. As step 218, the at least one sensor 24 monitors and/or senses the present health data of the user. If there is no abnormal health event, at step 220, the wearable device 10 and/or the application 20 does not generate and/or transmit the at least one alert communication. Contrarily, at step 222, when an abnormal health event occurs, the wearable device 10 and/or the application 20 generates and/or transmits, via the communication interface 38, the at least one alert communication to the at least one predetermined contact. In certain embodiments, the at least one alert communication bypasses any device settings (e.g., silence and/or low volume) of the electronic device or other computing device of the at least one predetermined contact. At step 224, the at least one predetermined contact may be requested to acknowledge receipt of the at least one alert communication. If the at least one predetermined contact does not acknowledge receipt, step 218 is repeated. At step 226, when the at least one predetermined contact acknowledges receipt, no further action is taken.

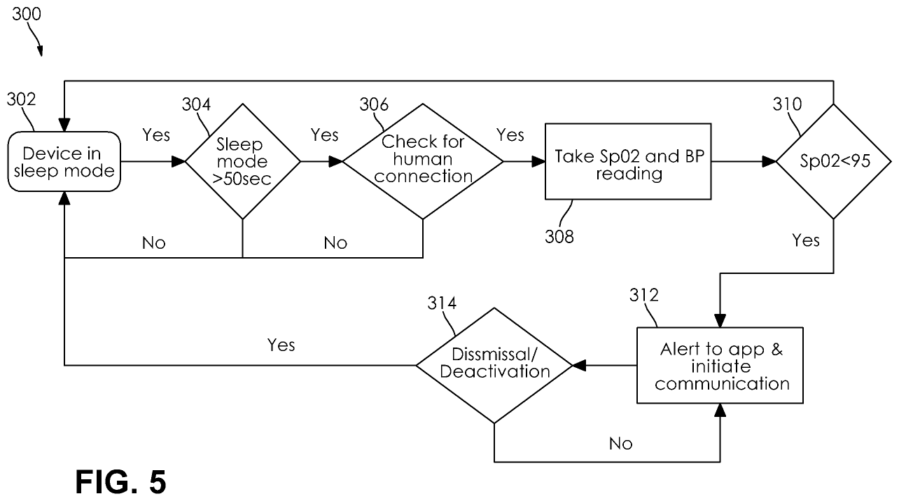

FIG. 5 is a flow diagram representing an exemplary method 300 of an operation process in accordance with various embodiments of the present disclosure. The method 300 may be performed by the wearable device 10 described with respect to FIGS. 1A-2.

At step 302, the wearable device 10 is in sleep mode. If the wearable device 10 has been in the sleep mode for a pre-determined period of time (e.g. 50 seconds) or less at step 304, the wearable device 10 remains in sleep mode at step 302. If the wearable device 10 has been in the sleep mode for longer than the pre-determined period of time, the wearable device 10 checks for a human connection (e.g., a hand or neck of a user) at step 306. If the human connection is not detected, the wearable device 10 returns to the sleep mode at step 302. If the human connection is detected, the wearable device 10 senses present health data (e.g., oxygen saturation levels and/or blood pressure (BP) levels of the user) at step 308. If the present health data at step 310, when compared to the stored health data levels/ranges, indicates that an abnormal health event has not occurred, then the wearable device 10 returns to the sleep mode at step 302. For instance, when the present oxygen saturation level is at or greater than the normal oxygen saturation level (e.g., when the present oxygen saturation level is at 95% or more). Alternatively, if the present health data at step 310, when compared to the stored health data levels/ranges, indicates that an abnormal health event has occurred, then the wearable device 10 and/or the application 20 may be further configured to provide at least one alert communication (e.g., a buzzer), via the input and output system 32, at step 312. If the user dismisses/deactivates (such as by pressing a button) the at least one alert communication at step 314, the wearable device 10 returns to the sleep mode at step 302. Alternatively, if the user does not dismiss/deactivate the at least one alert communication, the step 312 is repeated.

The purpose of the wearable device 10 is to notify the user and/or the predetermined contacts via the communication interface 38 (e.g., short message service (SMS) and/or phone call and/or application alert and/or email), when the wearable device 10 receives present health data outside of the stored health data and/or the algorithm parameters. The stored health data and/or the algorithm parameter can be adjusted and customizable to the application 20 and/or the wearable device 10. These present health data could include oxygen saturation (SpO2) and/or heart rate and/or heart rhythm and/or blood pressure and/or respiratory rate and/or and respiratory rhythm and/or respiratory depth.

Presently, there is not a wearable device that automatically notifies the user and/or the predetermined contacts via the communication interface 38 (e.g., SMS and/or phone call and/or application alert and/or email) when the user becomes incapacitated and/or showing present health data outside of the stored health data and/or the algorithm parameters. The user can become incapacitated for various medical conditions which could reflect on the present health data sensed and/or received by the wearable device 10. The wearable device 10 has an algorithm approach to alerting the user and/or the predetermined contacts to the possible need for assistance and/or emergency response when present health data specific to oxygen saturation (SpO2) and/or heart rate and/or heart rhythm and/or blood pressure and/or respiratory rate and/or and respiratory rhythm and/or respiratory depth is outside of the stored health data and/or the algorithm parameters.

One improvement of the wearable device 10 over the prior art is that the wearable device 10 will send to the user and/or the predetermined contacts an SMS and/or phone call and/or application alert and/or email when the present health data is outside of the stored health data and/or the algorithm parameters. The alert will be sent to the user and/or the predetermined contacts stored in the database of the wearable device 10.

The wearable device 10 may be well-suited for any person(s) at increased risk of becoming incapacitated such as by Covid, COPD, heart disease, substance abuse etc. One exemplary market would be substance abuser(s) at risk of overdose. Clinical changes to a person's vitals experiencing an overdose would potentially include a change (decrease) in oxygen saturation (SpO2) and/or heart rate and/or heart rhythm and/or blood pressure and/or respiratory rate and/or and respiratory rhythm outside of the stored health data and/or the algorithm parameters. In one scenario, the user of the wearable device 10 would become incapacitated after ingestion of a substance of abuse (e.g., an opiate). This could result in the user having a decreased respiratory rate and depth of breathing. Such decrease in oxygen saturation (SpO2) outside of the stored health data and/or the algorithm parameters would be recognized by the wearable device 10 and at least one alert communication would be sent via the communication interface 38 (e.g., SMS and/or phone call and/or application alert and/or email). This would provide the predetermined contacts an opportunity to initiate emergency response and/or response appropriate to the situation.

According to the National Institute on Drug Abuse, there were 91,799 overdose deaths reported in the United States in 2020 with an estimated 27.5 million people who have substance abuse/use problems, with 20.5 million adults in recovery. This population can benefit from timely notification of their change in present health data so the predetermined contacts can initiate emergency services and/or provide Narcan as a reversal agent to the ingested substance of abuse. As such, human lives could be saved by utilizing the wearable device 10 utilizing the algorithmic response when collecting present health data.

Maximizing an efficiency of notification to the predetermined contacts is achieved when the user of the wearable device is experiencing present health data outside of the stored health data and/or the algorithm parameters. This improved notification process will increase a likelihood and an efficiency of emergency response to an incapacitated person(s) and/or user experiencing a change in the present health data outside of the stored health data and/or the algorithm parameters.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods may be made within the scope of the present technology, with substantially similar results.

What is claimed is:

1. A wearable device, comprising:
a case;
at least one sensor at least partially disposed in the case and configured to detect health data of a user; and
at least one processor in communication with the at least one sensor, the at least one processor configured to receive the health data and transmit a communication to initiate a response from at least one predetermined contact based upon the health data.

2. The wearable device of claim 1, wherein the at least one processor is configured to utilize at least one algorithm parameter to determine an occurrence of an abnormal health event.

3. The wearable device of claim 2, wherein the at least one algorithm parameter is based upon a predisposition of a user.

4. The wearable device of claim 2, wherein the at least one processor is in communication with at least one storage device configured to store at least one of the health data, the at least one algorithm parameter, and personal information of the at least one predetermined contact.

5. The wearable device of claim 2, wherein the wearable device is configured to use machine learning to at least one of generate and adjust the at least one algorithm parameter.

6. The wearable device of claim 1, wherein the at least one sensor is configured to detect the health data at one or more of a predetermined frequency, a customizable frequency, and on demand.

7. The wearable device of claim 1, wherein the at least one processor is configured to generate at least one alert communication via an input and output system of the wearable device during an occurrence of an abnormal health.

8. The wearable device of claim 1, further comprising a graphical user interface in communication with the at least one processor and an input and output system of the wearable device.

9. The wearable device of claim 1, wherein the communication transmitted by the at least one processor is configured to bypass at least one setting of an electronic device of the at least one predetermined contact.

10. The wearable device of claim 1, wherein the at least one sensor is configured to detect a human connection.

11. A method of initiating a response, comprising steps of:
providing a wearable device including at least one sensor and at least one processor;
detecting health data of a user via the at least one sensor; and
transmitting a communication to initiate a response from at least one predetermined contact based upon the health data.

12. The method of claim 11, further comprising the step of determining, via the at least one processor, an occurrence of an abnormal health event using at least one algorithm parameter.

13. The method of claim 12, wherein the at least one algorithm parameter is based upon a predisposition of a user.

14. The method of claim 11, further comprising the step of storing at least one of the health data, at least one algorithm parameter, and personal information of the at least one predetermined contact.

15. The method of claim 11, further comprising the step of using machine learning to at least one of generate and adjust at least one algorithm parameter.

16. The method of claim 11, wherein the at least one sensor is configured to detect the health data at one or more of a predetermined frequency, a customizable frequency, and on demand.

17. The method of claim 11, further comprising the step of outputting at least one alert communication via an input and output system of the wearable device during an occurrence of an abnormal health event.

18. The method of claim 11, wherein the wearable device further includes a graphical user interface in communication with the at least one processor and an input and output system of the wearable device.

19. The method of claim 11, wherein the communication transmitted by the at least one processor is configured to bypass at least one setting of an electronic device of the at least one predetermined contact.

20. The method of claim 11, further comprising the step of detecting detect a human connection using the at least one sensor.
